(19) United States
(12) Patent Application Publication
(10) Pub. No.: US 2017/0330485 A1
(43) Pub. Date: Nov. 16, 2017

(54) NEURO-DRILL-STENCIL TRAINER

(71) Applicants: Indian Council of Medical Research, New Delhi (IN); All India Institute of Medical Sciences, New Delhi (IN); Indian Institute of Technology, New Delhi (IN); Department of Science & Technology, New Delhi (IN)

(72) Inventors: Ramandeep Singh, New Delhi (IN); Baby Britty, New Delhi (IN); Vinkle Srivastav Kumar, New Delhi (IN); Ashish Suri, New Delhi (IN); Subhashis Banerjee, New Delhi (IN); Prem K. Karla, New Delhi (IN); Sanjiva Prasad, New Delhi (IN); Subodh Kumar, New Delhi (IN); Kolin Paul, New Delhi (IN); Sneth Anand, New Delhi (IN)

(21) Appl. No.: 15/517,773

(22) PCT Filed: Oct. 8, 2015
(86) PCT No.: PCT/IN2015/000380

§ 371 Date: Apr. 7, 2017

(30) Foreign Application Priority Data
Oct. 8, 2014 (IN) 2874/DEL/2014

(51) Int. Cl. G09B 23/28; A61B 17/17

(52) U.S. Cl. CPC G09B 23/28; A61B 17/17

(57) ABSTRACT

A neuro-drill stencil trainer for demarcation of drilling on flat and curved bones comprises: i) one flat stencil which further comprises: a flat base (5), outer region (1), inner region (2), four slots (3) to define constrained region for drilling procedure, a handle (4) placed on the center region, to firmly hold the stencil at stationary position and ii) one curved stencil further comprises curved base (6), outer circles (7), inner circles (8), slots (9) and handle (10).

### FIELD OF INVENTION

The present invention relates to a neuro-drill-stencil-trainer for training of drilling procedure in neurosurgery and more specifically to provide demarcation on flat and curved bones to evaluate the proficiency of the procedure.

### BACKGROUND AND PRIOR ART OF THE INVENTION

Neurosurgery is the youngest surgical specialty that demands for effective training and education for developing the precise skills. Drilling is an important task performed to access the deeper structures of the brain by removing the bone flaps of outer skull. Drilling is also performed on inner bones during different surgical procedures and is usually done under magnification. Neurosurgery drilling tools have evolved significantly from hand driven drills to high-speed electric/pneumatic drills. These high-speed drills should be operated with high precision and control. Any error while operating the drill may lead to devastating consequences because they are pressed against the surface at rates of hundred thousand revolutions per minute. Therefore effective training is required to avoid skidding of drills, rupture of dura mater and to save critical structures from damage.

The usual drilling practice is imparted on flat and curved bones under naked eye and advanced training is practiced under microscope with defined magnification. Defined workspace for performing drilling task is essential for precise training in neurosurgery. Therefore, training requires manipulation in fine and constrained area to limit the hand and tool motions. There is no standard marking and evaluation procedure for drilling in neurosurgery.

U.S. Pat. No. 5,954,687 provided a burr hole ring with catheter for use as an injection port that relates to a burr hole ring with a catheter for use as an injection port comprises a modified burr hole ring adapted to engage the skull at a burr hole drilled therein. The interior of the burr hole ring defines a fluid reservoir that may be accessed by a needle or stylet inserted through a septum positioned over the top of the burr hole ring. The reservoir is in fluid communication with the central lumen of a catheter. The assembly comprises a fluid flow path suitable for the transfer of fluids to or from selected location at, near or within the brain. A filter layer may be provided to prevent contamination of the drug infusion site.

US App. No. 20100041004 A1 provided a surgical burr hole drilling simulator that relates to a system, method and device for simulating a medical procedure include a haptic mechanism controllable to provide feedback to a user manipulating a medical device. In an embodiment, a pair of three degree of freedom haptic devices is coupled to provide six degree of freedom measurement and force/moment feedback to the user. The six degree of freedom haptic device may be configured to provide different resolutions for different degrees of freedom, depending on simulation requirements. In an embodiment, a load cell is used to provide higher resolution in a dimension determined to be of greater criticality to realistic simulation.

U.S. Pat. No. 6,328,565 B1 disclosed a training method and apparatus for dental bur identification that relates to a bur block adapted for identifying and training dental personnel employs a block and a cover. The block has a surface having a number of holes sized to retain a number of dental burs. Each hole has indicia representing characteristics of the bur situated in the hole. The characteristics include sizes and shapes of the bur, colors, grits, part numbers, and the like. By putting the characteristics of each bur in proximity to the bur itself, persons can readily learn and/or become accustomed to the bur characteristics so that bur identification in the future is made easier.

U.S. Pat. No. 4,821,716 A disclosed a drilling an orifice in the skull that relates to an apparatus and method for drilling an orifice in a human cranium at an angle of substantially 90° to a plane defined by a tangent to the surface of the cranium at the orifice. The apparatus comprises a first tubular guide for directing a drill at the proper angle, the first guide being supported upon the cranium by a plurality of leg members. The invention further comprises a hand-operated twist drill device, configured for insertion into the tubular guide. A second guide, of reduced diameter relative to the first tubular guide is insertable into the first guide upon completion of the burr hole to facilitate entry of a catheter into the ventricular portion of the patient's brain. The method of the invention comprises positioning the first drill guide upon the patient's cranium, inserting the twist drill therein so as to prepare a burr hole extending at an angle of substantially 90° to a plane tangential to the surface, replacing the drill with a catheter guide insertable within the drill guide and inserting a catheter through the second guide, through the burr hole and into a ventricular portion of the patient's brain.

However, it can be seen from the prior arts that trainers for high drilling surgical training under microscope are lacking.

Hence, the present invention provides a novel stencils for providing enclosed constrained environment to impart drilling training under microscope for neurosurgery. The said stencil is provided with markings for burr holes at defined constrained area and different angles of stencils to match the surface of flat and curved bones.

### Object of the Invention

It is therefore an object of the present invention is to provide simple stencil for precisely demarcating the constrained region of drilling procedure.

Another object of the present invention is to help the evaluation of effectualness of the drilling procedure.

Yet another object of the present invention is to provide a stencil that is easy to use on flat as well as curved surfaces.

### SUMMARY OF THE INVENTION

According to this present invention, there is provided neuro-drill stencil trainer for demarcation of drilling on flat and curved bones comprises: i) one flat stencil which further comprises: a flat base (5), outer region (1), inner region (2), four slots (3) to define constrained region for drilling procedure, a handle (4) placed on the center region to firmly hold the stencil at stationary position and ii) one curved stencil further comprises curved base (6), outer circles (7), inner circles (8), slots (9) and handle (10);
said stencil is made up of rubber like polymer for providing demarcation of burr holes for drilling;
said slots (3) are rectangular in shape of 24×2 mm size;
said outer region further comprises four holes (1) of 3 mm diameter at the four corners to provide reference markings for evaluation of the performed activity;
said inner region further comprises of four holes (2) each having 2.5 mm diameter specify the location for making burr holes;
said handle (4 and 10) comprises a flat base design for flat bones and a curved base design for curved bones so that proper contact on the surface of bone can achieved;
the method of drilling with the neuro drill stencil trainer comprises producing burr-holes on the inner four markings by performing circular and semicircular motions of the drill bit and the rectangular slots are made in between these burr holes starting from top left to top right, top right to bottom right, bottom right to bottom left and bottom left to top left directions;
said drilling is performed in the diagonal direction starting from top left to bottom right marking and followed by bottom left to top right;
the method of drilling with the neuro drill stencil trainer can be recorded using a microscope and subjected to offline analysis.

### BRIEF DESCRIPTION OF THE INVENTION

According to this invention there is provided a stencil for providing demarcation in the flat and curved bones to impart training under microscope for drilling procedure in neurosurgery has been designed. The stencil is formed with an outer region made up of four circles and four grooves to mark the square region within which drilling is to be performed. It also has an inner region made of four circles, which are used to mark the spots to indicate the burr-hole positions. A handle is provided to hold the stencil at a stationary position related to the bone during marking. It is having a flat base design for flat bones and curved (convexo-concave) base design for curved bones for proper contact on to the bone surface.

### BRIEF DESCRIPTION OF THE ACCOMPANYING DRAWINGS

FIG. 1 is an isometric view of the flat stencil

FIG. 2 is the top view of the flat stencil

FIG. 3 is the isometric view of the curved stencil

FIG. 4 is the top view of the curved stencil

### DETAILED DESCRIPTION OF THE ACCOMPANYING DRAWINGS

The present invention relates to microscopic high speed drilling training for neurosurgical procedures. One aspect of the invention is to provide rubber stencils for providing demarcation of burr holes for drilling with diamond match head and cutting match head drill bits of brand X on curved and flat bones. Another aspect of the invention is provide a constrained operating area to the trainees and to practice precise, straight and controlled movements of drill bit under microscopic magnification.

Embodiment consists of a demarked outer region for evaluation of the performed activity. The activity is constrained in this region and consists of making four burr holes, four edge lines and two diagonal lines. Yet another aspect of the invention is to record the performed activity using microscope output of brand Y on a computer. These recordings can be used for offline analysis of the effectiveness of performed activity.

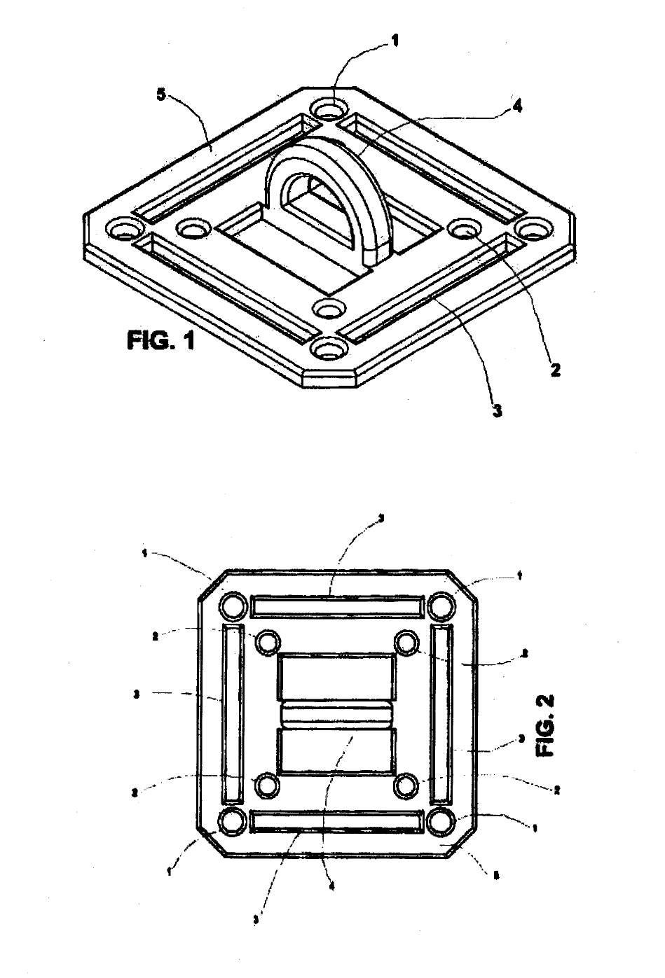

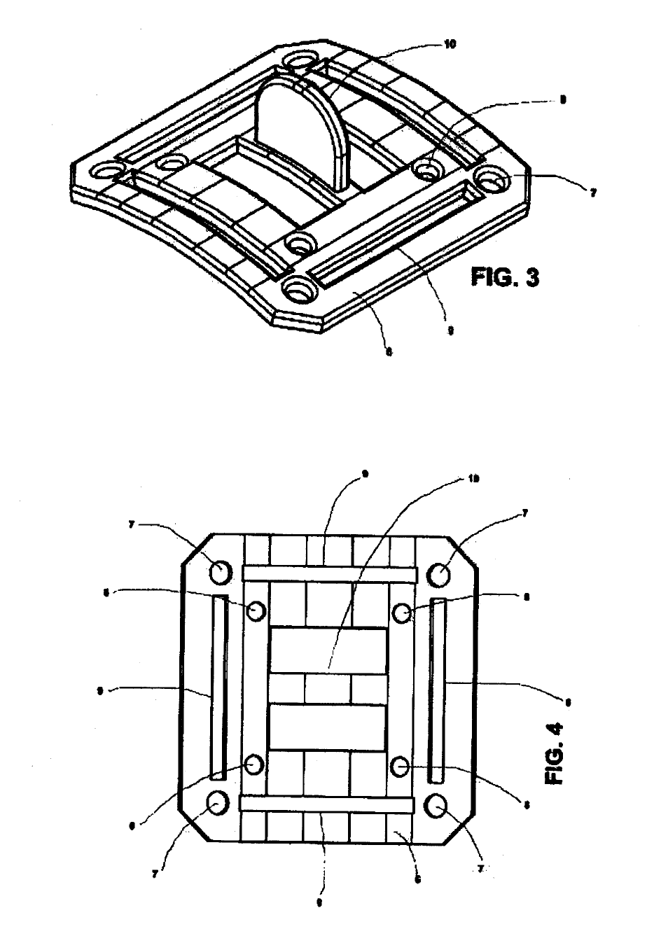

FIG. 1, depicts the isometric view of the flat stencil made up of rubber like polymer with flat base 5, outer circles 1, inner circles 2, slots 3 and handle 4. FIG. 2 shows the top view of flat stencil with outer region comprising of four rectangular slots 3 of 24×2 mm to define constrained region in which the drilling procedure is to be performed, four holes 1 of 3 mm diameter at the four corners to provide reference markings for evaluation of the performed activity. The inner region of stencil comprises of four holes 2 of 2.5 mm diameter to specify the location to make burr holes on the curved and flat bones. A handle 4 is placed on the center region to firmly hold the stencil, while marking. FIG. 3 depicts the isometric view of stencil curved at 10 degrees to match the curvature of curved bones. Similarly FIG. 4 shows the top view of curved stencils depicting with curved base 6, outer circles 7, inner circles 8, slots 9 and handle 10.

Drilling procedure is divided into three activities: burr hole activity, edge line activity and diagonal line activity. Match head cutting drill bit is used for rough drilling at lower magnification (4.25×) and match head diamond drill bit is used for fine drilling at higher magnification (7.08×) of the operating microscope. Procedure starts by making burr holes on the inner four markings by performing circular and semicircular motions of the drill bit. Then rectangular slots are made in between these burr holes starting from top left to top right, top right to bottom right, bottom right to bottom left and bottom left to top left directions. Then the drilling is performed in the diagonal direction starting from top left to bottom right marking and followed by bottom left to top right.

We claim:

1. A neuro-drill stencil trainer for demarcation of drilling on flat and curved bones comprises:
i) one flat stencil which further comprises:
a flat base (5), outer region (1), inner region (2), four slots (3) to define constrained region for drilling procedure, a handle (4) placed on the center region to firmly hold the stencil at stationary position and
ii) one curved stencil further comprises curved base (6), outer circles (7), inner circles (8), slots (9) and handle (10).

2. The neuro-drill stencil trainer for demarcation of drilling on flat and curved bones as claimed in claim 1, wherein said stencil is made up of rubber like polymer for providing demarcation of burr holes for drilling.

3. The neuro-drill stencil trainer for demarcation of drilling on flat and curved bones as claimed in claim 1, wherein said slots (3) are rectangular in shape of 24×2 mm size.

4. The neuro-drill stencil trainer for demarcation of drilling on flat and curved bones as claimed in claim 1, wherein said outer region further comprises four holes (1) of 3 mm diameter at the four corners to provide reference markings for evaluation of the performed activity.

5. The neuro-drill stencil trainer for demarcation of drilling on flat and curved bones as claimed in claim 1, wherein said inner region further comprises of four holes (2) each having 2.5 mm diameter specify the location for making burr holes.

6. The neuro-drill stencil trainer for demarcation of drilling on flat and curved bones as claimed in claim 1, wherein said handle (4 and 10) comprises a flat base design for flat bones and a curved base design for curved bones so that proper contact on the surface of bone can achieved.

7. The method of drilling with the neuro drill stencil trainer as claimed in claim 1, comprises producing burr-holes on the inner four markings by performing circular and semicircular motions of the drill bit and the rectangular slots are made in between these burr holes starting from top left to top right, top right to bottom right, bottom right to bottom left and bottom left to top left directions.

8. The method of drilling with the neuro drill stencil trainer as claimed in claim 7, wherein drilling is performed in the diagonal direction starting from top left to bottom right marking and followed by bottom left to top right.

9. The method of drilling with the neuro drill stencil trainer as claimed in claim 7, can be recorded using a microscope and subjected to offline analysis.

10. The neuro-drill stencil trainer or demarcation of drilling on flat and curved bones as claimed in claim 1, as disclosed herein is supported by the accompanying drawings.
